(19) United States
(12) Patent Application Publication
(10) Pub. No.: US 2018/0135038 A1
(43) Pub. Date: May 17, 2018

(54) IMMOBILIZED PLASMINOGENASE COMPOSITION, PREPARATION PROCESS, USE AND DEVICE COMPRISING SUCH A COMPOSITION

(71) Applicant: ARCADOPHTA, Toulouse (FR)

(72) Inventors: Gerard REBOUL, Toulouse (FR); Carine SAUX, Toulouse (FR); Francoise OUARNE, Castanet Tolosan (FR)

(21) Appl. No.: 15/566,642

(22) PCT Filed: Apr. 14, 2016
(86) PCT No.: PCT/FR2016/050869

§ 371 Date: Oct. 13, 2017

(30) Foreign Application Priority Data
Apr. 15, 2015 (FR) 15 53347

(51) Int. Cl. C12N 11/08; C12N 9/72; A61K 35/16; A61K 38/48

(52) U.S. Cl. CPC C12N 11/08; C12N 9/6456; A61K 38/484; A61K 35/16; C12Y 304/21

(57) ABSTRACT

Disclosed is an enzymatic composition including:—at least one enzyme, termed plasminogenase, for converting, into plasmin, plasminogen from a blood plasma medium including plasminogen;—a solid support that is insoluble in aqueous solution, the solid support having dimensions suitable for being able to be retained on a filter having a cut-off threshold of less than or equal to 0.22 μm. The plasminogenase is bound to the solid support and remains bound to this support on contact with a blood plasma medium. The composition is in the dry state. Also disclosed is a process for preparing such an enzymatic composition, to the use thereof and to a device (20) for preparing a blood plasma medium ex vivo rich in sterile plasmin and free of enzymatic composition.

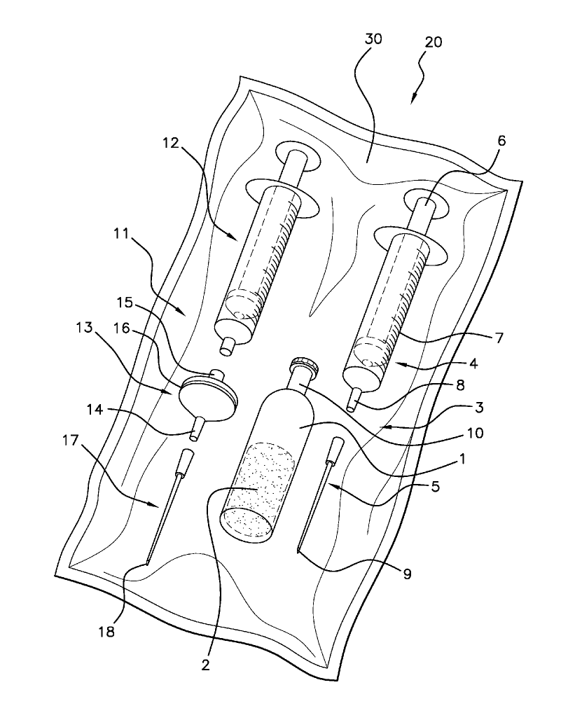

The invention relates to an enzymatic composition comprising a plasminogenase immobilized on a solid support, to a process for preparing such an enzymatic composition, to the use of such an enzymatic composition for the preparation of a plasmin-rich autologous ex-vivo plasma medium and to a device comprising such an enzymatic composition.

The invention relates in particular to an enzymatic composition and a device comprising such an enzymatic composition which are suitable for use in the context of a treatment of a pathology in a patient in which an autologous composition enriched in autologous plasmin is required, for example in the context of a cardiovascular pathology or in the context of a surgical intervention, in particular during an intravitreal surgical intervention in ophthalmology.

Certain pathological indications in ophthalmology such as vitreomacular traction (VMT) give rise to macular holes created by differential tangential traction stresses arising between the vitreous body and the retina. Treatment of these pathologies necessitates relieving the retina from these stresses. The known treatments are mainly of surgical nature directed toward mechanically separating the retina and the vitreal body.

WO 2010/125148 discloses a treatment for tractional retinal detachment in which a sterile composition obtained by addition of heterologous urokinase to a patient's plasma is administered to the patient's vitreous humour. This sterile composition comprises heterologous urokinase and is liable to pose a problem of immunological and inflammatory reaction in the patient.

An injectable composition comprising a recombinant protein formed from the catalytic domain of plasmin and the effect of injecting such an injectable composition at the vitreo-retinal interface of human eyes ex vivo or of cats' eyes in vivo is also known (Gondorfer et al. (2004), Investigative Ophthalmology & Visual Science, 45; 2, 641-647. Posterior Vitreous Detachment Induced by Microplasmin).

Such a recombinant protein is, however, complex to manufacture. The presence of heterologous recombinant protein in such an injectable composition leads to an immune reaction problem in the patient receiving this injectable composition. In addition, the cost of this injectable composition is high, and so alternative solutions to the use of recombinant proteins are sought, with which the risks of immune reaction would also be minimized.

A process for the conversion into plasmin of plasminogen extracted beforehand from plasma is also disclosed in JP2007/068497. JP2007/068497 does not teach a process for converting into plasmin, directly in a plasma, plasminogen from the plasma and for preparing a plasmin-enriched autologous plasma.

The invention is thus directed toward solving all of these problems.

In particular, the invention is directed toward proposing an enzymatic composition, a process for preparing and the use of such an enzymatic composition, and a device comprising such an enzymatic composition allowing the ex-vivo conversion of the plasminogen of a blood plasma medium into plasmin under the effect of the enzymatic composition. The invention makes it possible to prepare a plasmin-rich ex-vivo plasma medium free of heterologous enzyme or containing only a minimal residual amount of free heterologous enzyme which in particular is insufficient to bring about an immune reaction when said plasmin-rich ex-vivo plasma medium is placed in contact—especially by injection—with a patient's tissue(s).

The invention is directed toward proposing an enzymatic composition, a process for preparing and the use of such an enzymatic composition, and a device comprising such an enzymatic composition, which are suitable for allowing rapid conversion—especially in a time of between 15 minutes and 60 minutes at 37° C.—of plasminogen of a blood plasma medium into plasmin.

To do this, the invention relates to an enzymatic composition comprising:
at least one enzyme, named plasminogenase, for converting into plasmin the plasminogen of a blood plasma medium comprising plasminogen;
a solid support that is insoluble in aqueous solution,

the solid support having dimensions suitable for allowing it to be retained on a filter with a cut-off threshold of less than or equal to 0.22 μm;

characterized in that said plasminogenase is linked to the solid support and remains linked to this support on contact with a blood plasma medium, and in that the composition is in dry form.

Throughout the text, the following terminology is adopted:
the term “blood plasma medium” denotes any liquid medium that is free of viable blood cells and that results directly from a fractionation treatment of liquid, non-coagulated blood—especially human blood—under conditions suitable for allowing separation of the blood cells (erythrocytes, leukocytes, platelets) and of the blood plasma medium that is free of viable blood cells. Such a separation may be performed, for example, by centrifugation or by cell sorting in a microfluidic process;
the term “ex-vivo plasma medium” denotes any blood plasma medium extracted from the human or animal body;
the term “plasminogenase” denotes any enzyme with activity for converting plasminogen into plasmin by cleaving a peptide bond of plasminogen; and
the term “stable bond” denotes all of the forces which ensure, under predetermined conditions, cohesion between groups of atoms, in particular between the solid support and at least one plasminogenase when the enzymatic composition is placed in contact by immersion at a temperature of about 37° C. in a blood plasma medium;
the term “substantially” indicates, as is usual, that a structural characteristic—such as a value—or a functional characteristic, must not be taken as marking an abrupt discontinuity, which would have no physical meaning, but covers not only this structure or this function, but also slight variations of this structure or of this function which produce, within the technical context under consideration, an effect of the same nature, or else of the same degree.

The invention relates to an enzymatic composition comprising at least one plasminogenase immobilized on a solid support in divided form, said enzymatic composition being suitable to be able:
to be placed in contact with a blood plasma medium;
to allow conversion into plasmin of at least some of the plasminogen of the blood plasma medium under the action of said plasminogenase in contact with the blood plasma medium;
to be separated from the plasmin-enriched blood plasma medium by filtration on a membrane or filter with a cut-off threshold of less than or equal to 0.22 μm.

The plasminogenase, the solid support and the bond formed between the plasminogenase and the solid support are chosen so that the plasminogenase remains linked to the solid support when the enzymatic composition is placed in contact with a blood plasma medium.

The enzymatic composition according to the invention is in dry form. The term “dry form” in particular reflects the fact that the enzymatic composition according to the invention does not provide to the blood plasma medium with which it is placed in contact any liquid, especially any aqueous liquid, which is liable to modify the ionic composition and/or the ionic concentrations of the blood plasma medium with which the enzymatic composition is placed in contact. It therefore does not substantially modify the osmotic properties of the blood plasma medium with which it is placed in contact. Nor does it modify, by its sole addition to the blood plasma medium, the concentration of the plasminogen present in the blood plasma medium to which it is added.

The enzymatic composition according to the invention does not release into the blood plasma medium placed in contact with the enzymatic composition any plasminogenase in free form, or releases into the blood plasma medium placed in contact with the enzymatic composition only a small amount of plasminogenase in free form. The enzymatic composition according to the invention is thus able to be used in a process for preparing a plasmin-rich ex-vivo plasma medium that is substantially free of heterologous plasminogenase, from a blood plasma medium formed from blood collected from a patient. Such a plasmin-rich autologous ex-vivo plasma medium substantially free of heterologous plasminogenase is intended to be used in this same patient.

The inventor has observed that it is possible to immobilize at least one plasminogenase on a solid support in divided form that is insoluble in aqueous solution while at the same time conserving plasminogenase activity. Such an enzymatic composition allows, firstly, efficient conversion of at least some of the plasminogen of a blood plasma medium into plasmin and, secondly, facilitated separation of the enzymatic composition and of a plasmin-rich ex-vivo plasma medium obtained by placing a blood plasma medium and the enzymatic composition in contact, while limiting—in particular totally preventing—significant untimely release of plasminogenase into this plasmin-rich ex-vivo plasma medium. The plasmin-rich ex-vivo plasma medium is thus substantially free of exogenous and heterologous plasminogenase.

Advantageously and according to the invention, the solid support is in divided form and formed from particles having three dimensions extending in three mutually orthogonal directions, at least two of the three dimensions being greater than 0.22 μm.

Advantageously and according to the invention, each dimension of the particles is greater than 0.22 μm.

Advantageously and according to the invention, at least two of the dimensions of the particles of the solid support in divided form are between 1 μm and 500 μm, especially between 10 μm and 500 μm, preferably between 100 μm and 500 μm. Advantageously and according to the invention, each dimension of the particles of the solid support in divided form is between 1μm and 500 μm, especially between 10 μm and 500 μm, preferably between 100 μm and 500 μm. Advantageously, the dimensions of the particles of the solid support in divided form are chosen so that the enzymatic composition is retained on a sterilizing filter, i.e. on a filter with a cut-off threshold of less than or equal to 0.22 μm.

Advantageously and according to the invention, the solid support is formed from a porous material. Advantageously and according to the invention, the porous material has pores with a mean diameter of between 5 nm and 50 nm, preferably between 10 nm and 20 nm. Advantageously, the porous material is chosen from the group formed from rigid materials. Advantageously and according to the invention, the solid support is formed from a material chosen from the group formed from materials of high specific surface area.

Advantageously and according to a particular variant of the invention, the solid support for the enzymatic composition is a solid support with the exclusion of the solid supports mentioned in JP2007/068497. The solid support for the enzymatic composition according to the invention is a solid support with the exclusion of the support Sepharose 4FF/BB-CNBr, the support Sepharose 4FF-NHS, the support Sepharose 4B-ECH, the support Affi-Prep 10, the support Reacti-Gel, the support Sepharose 6B-Epoxy, the support Sepharose 4B EAH, the support Sepharose 6B-Thiopropyl, the support Sepharose-thiol, the support Affi-Prep-Hz, the support TNB-thiol and ap-chloromercuribenzoate support.

Advantageously and according to the invention, the solid support—especially the solid support in divided form—is formed from a material chosen from the group formed from polyglucoside polymers and polymethacrylic polymers.

Advantageously and according to another particular variant of the invention, the solid support is chosen from the group formed from the supports Sepabeads® EC-HFA/S and GE Healthcare Epoxy-Activated Sepharose™. Advantageously, the solid support in divided form is formed from spherical particles with a mean diameter of between 100 μm and 300 μm.

Advantageously and according to the invention, the solid support—especially the solid support in divided form—is formed from a material chosen from the group formed from polymethacrylic polymers. Advantageously and according to the invention, the solid support is formed from a material chosen from the group formed especially from polymethacrylic copolymers, for example poly(glycidyl methacrylic (GMA)/ethylene dimethacrylic (EDMA) copolymers.

Advantageously and according to the invention, at least one plasminogenase—especially each plasminogenase—is a serine endopeptidase—especially an endopeptidase with antithrombotic activity—of the class EC 3.4.21 of the enzyme classification.

Advantageously and according to the invention, at least one plasminogenase—especially each plasminogenase—is chosen from the group formed from a urokinase, a streptokinase, a nattokinase and a tissue plasminogen activator (t-PA). Advantageously and according to the invention, at least one plasminogenase is the urokinase U0633 (Sigma-Aldrich, Lyons, France). Advantageously and according to the invention, the enzymatic composition comprises a single plasminogenase. As a variant, the enzymatic composition may comprise a plurality of different plasminogenases as a mixture.

Advantageously and according to the invention, at least one plasminogenase is linked to the solid support via at least one stable bond chosen from the group formed from a covalent bond, an ionic bond, a covalent coordination bond (or dative bond) and a hydrophobic interaction of van der Waals type.

Advantageously and according to the invention, at least one stable bond is chosen to withstand contact with an aqueous NaCl solution at a concentration of 0.5 M.

Advantageously and according to the invention, at least one plasminogenase—especially each plasminogenase—is linked to the solid support via at least one covalent bond. In particular, at least one such covalent bond is formed by chemical reaction between a free amine group of said plasminogenase and an epoxy group of the solid support.

Advantageously and according to alternative embodiments of the invention, the enzymatic composition comprises a single plasminogenase or a mixture of a plurality of plasminogenases.

Advantageously and according to the invention, the enzymatic composition is suitable for forming, by placing in contact an enzymatic composition according to the invention and a blood plasma medium, a plasmin-rich ex-vivo plasma medium that is substantially apyrogenic. An enzymatic composition according to the invention is adapted so as not to release any heterologous compound—especially any heterologous plasminogenase—into a plasmin-rich ex-vivo plasma medium obtained by placing in contact the enzymatic composition and a blood plasma medium, especially at a temperature of about 37° C., said heterologous compound being capable of inducing an immune reaction in an individual treated with an amount of this plasmin-rich ex-vivo plasma medium.

The pyrogenic/apyrogenic nature of the plasmin-rich ex-vivo plasma medium obtained by placing in contact a blood plasma medium and an enzymatic composition according to the invention is analysed by measuring the body temperature of rabbits which have received an injection of plasmin-rich autologous ex-vivo plasma medium. The absence of increase of the rabbit's body temperature following the injection reflects the apyrogenic nature of the plasmin-rich ex-vivo plasma medium.

Advantageously and according to the invention, the enzymatic composition is free of any microbial germs, in particular of any pathogenic microbial germs. Advantageously and according to the invention, the enzymatic composition is sterile.

The enzymatic composition is in dry form. It may be in the form of a dry powder suitable for placing directly in contact with a blood plasma medium, especially without addition of water or of aqueous solution (such as water for an injectable solution or physiological saline) and for allowing conversion into plasmin of at least some of the plasminogen of the blood plasma medium under the action of said plasminogenase in contact with the blood plasma medium.

The enzymatic composition is in dry and dehydrated form; it may thus be conserved in dry and dehydrated form; it is suitable for placing directly in contact with a blood plasma medium, especially without any addition of water or of aqueous solution (such as water for an injectable solution or physiological saline) and for converting into plasmin at least some of the plasminogen of the blood plasma medium under the action of said plasminogenase in contact with the blood plasma medium.

Advantageously and according to the invention, the enzymatic composition is in the form of a dehydrated powder.

Advantageously and according to the invention, each plasminogenase is linked to the solid support so as only to introduce, into a blood plasma medium with which the enzymatic composition is placed in contact, a mass of free plasminogenase of less than 400 μg, said contact being performed according to the process below:
a mass of between 0.01 g and 0.5 g, especially about 0.1 g, of enzymatic composition in dehydrated form is mixed at a temperature of about 37° C. with a volume of between 0.5 mL and 1.0 mL, especially about 0.7 mL, of blood plasma medium; and then
the contact is maintained for a time of more than 5 minutes, especially between 5 minutes and 60 minutes; and then
the enzymatic composition and the blood plasma medium are separated by filtration on a filter with a cut-off threshold of less than or equal to 0.22 μm; and
the mass of plasminogenase released into the blood plasma medium is measured.

Advantageously and according to the invention, the mass of free plasminogenase in the blood plasma medium with which the enzymatic composition is placed in contact is less than 200 μg, especially less than 100 μg, preferably less than 50 μg. Advantageously and according to the invention, the mass of free plasminogenase in the blood plasma medium with which the enzymatic composition is placed in contact is less than 40 μg, especially less than 30 μg, in particular less than 25 μg, preferably less than 20 μg, more preferentially less than 10 μg.

Advantageously and according to the invention, the mass of free plasminogenase in the blood plasma medium with which the enzymatic composition is placed in contact is less than 20 μg, especially less than 15.0 μg, in particular less than 8.0 μg, preferably less than 3.0 μg, more preferentially less than 1.5 μg.

Advantageously and according to the invention, the mass of free plasminogenase in the blood plasma medium with which the enzymatic composition is placed in contact is less than 10 μg, especially less than 8.0 μg, in particular less than 6.0 μg, preferably less than 5.0 μg, more preferentially less than 2 μg. Advantageously and according to the invention, the mass of free plasminogenase in the blood plasma medium with which the enzymatic composition is placed in contact is less than 0.5 μg.

The plasminogenase released is assayed via any suitable method, for example by “ELISA” quantitative immunoenzymatic assay using a primary antibody specific for plasminogenase and a quantifiable secondary antibody and by establishing a calibration curve using solutions containing known amounts of plasminogenase in blood plasma medium.

It is also possible to detect activity of plasminogenase released into the plasmin-rich ex-vivo plasma medium via an indirect method by analysing the variation over time of the plasmin activity of the plasmin-rich ex-vivo plasma medium obtained after removal by filtration of the enzymatic composition. Under non-limiting plasminogen concentration conditions, the virtual stability of the plasmin activity reflects the absence of free plasminogenase in the plasmin-rich ex-vivo plasma medium, whereas a substantial increase in plasmin activity may reflect the presence of free plasminogenase in the plasmin-rich ex-vivo plasma medium.

The enzymatic composition according to the invention makes it possible to form a plasmin-rich ex-vivo plasma medium that is sparingly immunogenic.

Advantageously and according to the invention, at least one plasminogenase—especially each plasminogenase—is linked to the solid support via a group of atoms comprising a main chain of atoms bonded linearly to each other via covalent bonds (i.e. the chain having the largest number of atoms), the main chain having a number of atoms at least equal to 4, especially between 10 and 15 atoms.

Advantageously and according to the invention, the enzymatic composition has an activity, named plasminogenase activity, for conversion into plasmin of the plasminogen of a blood plasma medium with which it is placed in contact under the following conditions:
a mass of between 0.01 g and 0.5 g, especially about 0.1 g, of said enzymatic composition in dehydrated form is placed in contact at a temperature of about 37° C. for a time of more than 5 minutes, especially between 5 minutes and 60 minutes, with a volume of between 0.5 mL and 1.0 mL, especially about 0.7 mL, of blood plasma medium, and
a plasmin-rich ex-vivo plasma medium is formed, which has, after separation by filtration of the enzymatic composition and of the plasmin-rich ex-vivo plasma medium, an initial enzymatic activity, named plasmin activity, as measured via a para-nitroaniline release test of greater than 0.1 μmol, especially between 0.1 and 0.3 μmol, preferably about 0.2 μmol, of para-nitroaniline released per minute and per millilitre (mL) of plasmin-rich ex-vivo plasma medium, said release test consisting in:
   mixing in the plasmin-rich ex-vivo plasma medium maintained at a temperature of 37° C. a chromogenic substrate S-2251 of formula (I) below:

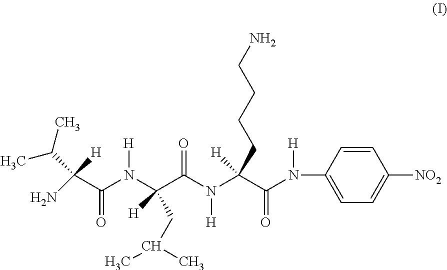 [SMILES: CC(C)C[C@H](NC(=O)[C@H](N)C(C)C)C(=O)N[C@@H](CCCCN)C(=O)Nc1ccc([N+](=O)[O-])cc1]

at an initial concentration of about 1 mM (i.e. 10⁻³ mol/L) in the plasmin-rich ex-vivo plasma medium,
evaluating, following the mixing, the initial rate of para-nitroaniline release (in μmol of para-nitroaniline) per minute and per millilitre (mL) of plasmin-rich ex-vivo plasma medium.

The plasmin activity of a plasmin-rich ex-vivo plasma medium obtained by placing in contact a blood plasma medium and an enzymatic composition according to the invention is measured via an indirect method in which an amount of enzymatic composition is placed in contact with an amount of blood plasma medium comprising plasminogen at 37° C. for a time of between 5 minutes and 60 minutes and under conditions that are suitable for allowing conversion of plasminogen of the blood plasma medium into plasmin and the formation of a plasmin-rich ex-vivo plasma medium.

A step of separating—especially by sterilizing filtration—the enzymatic composition and the plasmin-rich ex-vivo plasma medium formed by conversion of the plasminogen of the blood plasma medium into plasmin is then performed. Advantageously, it is possible to perform this sterilizing filtration step in a single filtration step on a filter with a cut-off threshold of less than or equal to 0.22 μm. However, it is also possible to perform this sterilizing filtration step in several steps comprising a first filtration for separation of the enzymatic composition and of the plasmin-rich ex-vivo plasma medium, the first filtration being non-sterilizing, and then a second filtration of the plasmin-rich ex-vivo plasma medium free of enzymatic composition, said second filtration being a sterilizing filtration on a filter with a cut-off threshold of less than or equal to 0.22 μm.

The plasmin activity of the plasmin-rich ex-vivo plasma medium is determined. In practice, a medium for measuring the plasmin activity is prepared in a spectrophotometric measuring cuvette by mixing at 37° C. a volume of plasmin-rich ex-vivo plasma medium and this same volume of an aqueous solution of a chromogenic substrate, for example S-2251 (H-D-Val-Leu-Lys-pNA.2HCl, Chromogenix, Le Pré-Saint-Gervais, France) of formula (IV) below:

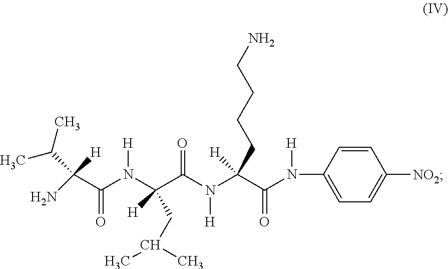 [SMILES: CC(C)C[C@H](NC(=O)[C@H](N)C(C)C)C(=O)N[C@@H](CCCCN)C(=O)Nc1ccc([N+](=O)[O-])cc1]

as chromogenic substrate at a concentration of about 2 mM (so that the concentration of S-2251 is about 1 mM in the measuring medium), which is capable of releasing para-nitroaniline under the action of plasmin of the plasmin-rich ex-vivo plasma medium. Starting from the time of mixing, the change in absorbance (optical density, OD₄₀₅ₙₘ) at 405 nm of the measuring medium maintained at 37° C. is measured over 5 minutes. The slope at the origin of the curve of change over time of the absorbance at 405 nm, i.e. the initial rate Vi of the reaction expressed in Δ_{abs}/min, is evaluated. The plasmin activity (expressed in U/mL of plasmin-rich ex-vivo plasma medium) is given by formula (II) below:

$$plasmin activity = \frac{Vi * Vm * {10}^{3}}{ɛ * L * Vp} , \qquad ( II )$$

in which:
Vi is the initial rate of the reaction expressed in Δ_{abs}/min;
Vm is the total volume (in mL) of the measuring medium;
ϵ is the molar extinction coefficient (in M⁻¹.cm⁻¹) of para-nitroaniline at 405 nm;
L is the optical path length (in cm) of the spectrophotometric measuring cuvette; and
Vp is the volume (in mL) of plasmin-rich ex-vivo plasma medium introduced into the spectrophotometric measuring cuvette.

Where appropriate, the basal plasmin activity of a blood plasma medium not enriched in plasmin is measured as a control by replacing in the above protocol the plasmin-rich ex-vivo plasma medium with the same volume of blood plasma medium from which the plasmin-rich ex-vivo plasma medium is derived. The basal plasmin activity value thus calculated is deducted from the plasmin activity value of the plasmin-rich ex-vivo plasma medium.

The invention also extends to a process for preparing an enzymatic composition according to the invention.

The invention also relates to a process for preparing an enzymatic composition according to the invention, in which:
at least one enzyme, known as plasminogenase, for converting into plasmin at least some of the plasminogen of a blood plasma medium comprising plasminogen is chosen;
a solid support that is insoluble in aqueous solution is chosen:
   which is suitable for forming with each plasminogenase a bond that is stable on contact with a blood plasma medium; and
   which has dimensions that are suitable for allowing it to be retained on a filter with a cut-off threshold of less than or equal to 0.22 μm; and
the solid support and each plasminogenase are placed in contact so as to link each plasminogenase to the solid support; and
a lyophilization step is performed so as to form the enzymatic composition.

Advantageously and according to the invention, each plasminogenase is immobilized on the solid support by simple placing in contact of each plasminogenase in liquid solution with the solid support. To do this, the solid support is immersed in a liquid solution—especially an aqueous solution—of each plasminogenase, which is kept stirring for a time sufficient to allow coupling between the solid support and each plasminogenase. A lyophilization step is then performed so as to form the enzymatic composition in dehydrated form and to conserve the activity of the plasminogenase(s).

Advantageously and according to the invention, conditions are chosen—especially temperature conditions—which are suitable for forming the enzymatic composition.

Advantageously and according to the invention, at least one plasminogenase—especially each plasminogenase—is chosen from the group formed from serine endopeptidases—especially endopeptidases with antithrombotic activity—of the class EC 3.4.21 of the enzyme classification.

Advantageously and according to a first embodiment of the invention, a solid support in divided form is chosen, formed from particles having three dimensions extending in three mutually orthogonal directions, at least two of said three dimensions being greater than 0.22 μm. Advantageously and according to the invention, each dimension of the particles is greater than 0.22 μm.

Advantageously and according to the invention, the solid support is chosen from the group of solid supports in divided form bearing particles of substantially spherical shape and with a diameter of greater than 0.22 μm. Such a solid support is thus able to be retained on filtration devices with a maximum permeation size of 0.22 μm.

Advantageously and according to the invention, at least two of the three dimensions of the particles of the solid support in divided form are between 1 μm and 500 μm, especially between 10 μm and 500 μm, preferably between 100 μm and 500 μm. Advantageously and according to the invention, each dimension of the particles of the solid support in divided form is between 1 μm and 500 μm, especially between 10 μm and 500 μm, preferably between 100 μm and 500 μm.

Advantageously and according to the invention, a solid support in divided form is chosen that is able to be retained by a filtering separation device with a cut-off threshold of about 0.22 μm, for example a filtering separation device comprising a polytetrafluoroethylene (PTFE) or polyvinylidene fluoride (PVDF) filter.

Advantageously and according to the invention, the solid support in divided form is formed from a material comprising at least one surface ligand that is capable of forming at least one stable bond—especially at least one covalent bond—with at least one plasminogenase. Advantageously, the surface ligand is formed from a group of atoms comprising a main chain of atoms bonded linearly to each other via covalent bonds (i.e. the chain with the largest number of atoms), the main chain having a number of atoms at least equal to 4, especially between 10 and 15.

Advantageously and according to the invention, at least one surface ligand comprises an epoxy group. Advantageously and according to the invention, at least one surface ligand is an amino-epoxide group linked to the solid support and of formula (III) below:

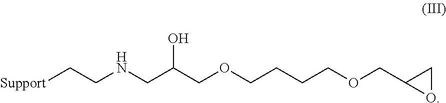

Advantageously and according to the invention, the material of the solid support—especially of the solid support in divided form—is chosen from the group formed from functionalized polyglycosides and functionalized methacrylate polymers. Advantageously and according to the invention, the material of the solid support in divided form is chosen from the group formed from polyglycosides surface-functionalized with at least one epoxy group and methacrylate polymers surface-functionalized with at least one epoxy group.

Advantageously, the solid support in divided form is chosen from the group formed from the supports Sepabeads® EC-HFA/S, the mean particle size of which is between 100 μm and 300 μm, and GE Healthcare Epoxy-Activated Sepharose™.

Advantageously, in a process according to the invention, at least one sterilization step is performed. At least one sterilization step is performed via any sterilization method known for being able to at least partially preserve the activity of plasminogenase. Advantageously and according to the invention, at least one step of sterilization by irradiation is performed.

Advantageously and according to the invention, at least one step of sterilization of the enzymatic composition is performed. Advantageously and according to the invention, at least one step of sterilization of the enzymatic composition by irradiation is performed. Advantageously and according to the invention, at least one step of sterilization by irradiation at a predetermined temperature is performed. Such a step of sterilization by irradiation may be performed at a temperature below 0° C. Advantageously and according to the invention, at least one step of sterilization by irradiation is performed at a temperature above 0° C.

Advantageously, in a process according to the invention, at least one step of sterilization by successive irradiations is performed, said successive irradiations being interrupted with at least one cooling phase.

Advantageously, at least one step of sterilization by irradiation releasing an amount of energy of between 5×10³ J/kg (5 kGy) and 5×10⁴ J/kg (50 kGy) is performed. Advantageously, at least one step of sterilization by irradiation with a radiation chosen from the group formed from β radiations and γ radiations is performed.

Advantageously and according to the invention, a step of lyophilization of the enzymatic composition is performed. An enzymatic composition in dehydrated powder form is formed.

Advantageously, in a process according to the invention, at least one step of sterilization by irradiation of the enzymatic composition after its lyophilization is performed. A process according to the invention makes it possible to obtain a sterile enzymatic composition.

In a process according to the invention, a sterile, apyrogenic enzymatic composition is obtained.

The invention also extends to an enzymatic composition that may be obtained via a process according to the invention.

The invention also extends to any use of an enzymatic composition according to the invention for the preparation of a sterile plasmin-rich ex-vivo plasma medium free of enzymatic composition.

The invention also extends to any use of an enzymatic composition according to the invention for the preparation of a plasmin-rich ex-vivo plasma medium, especially for the preparation of a sterile plasmin-rich ex-vivo plasma medium.

Advantageously and according to the invention, an enzymatic composition according to the invention is used to convert at least some of the plasminogen of a blood plasma medium into plasmin—which is the active form of plasminogen—and to form a plasmin-rich ex-vivo plasma medium without appreciable release of free plasminogenase into the plasmin-rich ex-vivo plasma medium and while limiting the risks of induction of an immune reaction in a patient to whom the plasmin-rich ex-vivo plasma medium has been administered. A plasmin-rich ex-vivo plasma medium is thus obtained which can be used in the context of any preventive or curative treatment of a pathology in which a transformation of plasminogenase into autologous plasmin is required, for example in the context of treating a cardiovascular pathology or in the context of a surgical intervention, in particular during an intravitreal intervention of a treatment—especially during a surgical intervention—of vitreomacular disorders in ophthalmology. Such a plasmin-rich ex-vivo plasma medium may be obtained in a sterile form, free of solid support and substantially free of enzyme, by separation/filtration on a sterilizing filter.

Advantageously and according to the invention, an amount of the enzymatic composition is placed and maintained in contact with an amount of the blood plasma medium comprising plasminogen for a time of between 15 minutes and 60 minutes, so as to form the plasmin-rich ex-vivo plasma medium comprising a mass of free plasminogenase of less than 25 μg per mL of plasmin-rich ex-vivo plasma medium. Advantageously, the enzymatic composition is maintained in contact with the blood plasma medium at a temperature close to the optimum temperature for plasminogenase, especially at a temperature of about 37° C. Such a process is simple to implement in as much as it requires only the placing in contact of the enzymatic composition and of the amount of blood plasma medium and maintenance of mixing at a predetermined temperature for a time that is suitable to allow conversion of plasminogen into plasmin under the effect of plasminogenase, followed by collection of the plasmin-rich ex-vivo plasma medium formed under the effect of the plasminogenase, said collection comprising filtration, especially sterilizing filtration. The plasmin-rich ex-vivo plasma medium is suitable for use directly, for example in intravitreal injection, during an intravitreal intervention of a treatment—especially during a surgical intervention—of vitreomacular disorders in ophthalmology. Advantageously, the plasmin-rich ex-vivo plasma medium is autologous.

Advantageously and according to the invention, an enzymatic composition according to the invention is used in a process performed in a room dedicated for performing intravitreal injection (IVT) and in which a competent person, especially a surgeon, an ophthalmologist or a healthcare person, performs:
a collection of the patient's blood; and then
the preparation—especially by centrifugation—of the blood plasma medium; and then
the placing in contact by mixing said blood plasma medium and the enzymatic composition according to the invention for a predetermined time and at a temperature of about 37° C.; and then
a separation of the plasmin-rich ex-vivo plasma medium and of the enzymatic composition for the purpose of injecting the plasmin-rich ex-vivo plasma medium into a patient's body.

Advantageously and according to the invention, the separation of the plasmin-rich ex-vivo plasma medium and of the enzymatic composition is performed by filtration using a filter that is capable of retaining the enzymatic composition.

Advantageously and according to the invention, the separation of the plasmin-rich ex-vivo plasma medium and of the enzymatic composition is performed by filtration using a filter with a cut-off threshold of about 0.22 μm . Advantageously, the separation of the plasmin-rich ex-vivo plasma medium and of the enzymatic composition is a sterilizing separation of the plasmin-rich ex-vivo plasma medium.

An enzymatic composition according to the invention is used in a treatment in which a plasmin-rich ex-vivo plasma medium is injected into a patient's vitreous body for the purposes of relieving the vitreomacular stresses by hydrolysis of protein fibres.

An enzymatic composition according to the invention is used for the preparation of a plasmin-rich ex-vivo plasma medium which is autologous—i.e. which is obtained from a blood plasma medium obtained from the blood of a patient and prepared for the purpose of injecting it into this sole patient—and which is free of heterologous protein, i.e. which is free of a sufficient amount of heterologous protein liable to induce an immune reaction in the patient.

Advantageously and according to the invention, the enzymatic composition is used for the preparation of a plasmin-rich ex-vivo plasma medium which is apyrogenic. The conditions for obtaining an apyrogenic plasmin-rich ex-vivo plasma medium are validated by measuring the body temperature of a rabbit which has received an injection of plasmin-rich ex-vivo plasma medium obtained from an amount of blood plasma medium of said rabbit. The absence of increase of the rabbit's body temperature following the injection reflects the apyrogenic nature of the plasmin-rich ex-vivo plasma medium and validates the conditions for obtaining it.

Advantageously and according to the invention, the enzymatic composition is used for the preparation of a plasmin-rich ex-vivo plasma medium which is sterile.

The invention also extends to a device for preparing a sterile plasmin-rich ex-vivo blood plasma medium which is free of enzymatic composition, comprising an amount of enzymatic composition according to the invention and a filter with a cut-off threshold of less than or equal to 0.22 μm.

The invention also extends to a device comprising:
a container—especially a hermetically closed container—containing the amount of enzymatic composition;
a device for introducing blood plasma medium into the container;
a device for collecting from the container a plasma medium formed in the container under the effect of the enzymatic composition;
the collecting device (11) and the filter being arranged to allow the filtration of the plasma medium and the production of a filtrate constituting a sterile plasmin-rich ex-vivo plasma medium free of enzymatic composition.

A device according to the invention is advantageously in the form of a kit, i.e. an assembly comprising several components in separate form, including at least:
the container containing an amount of a sterile enzymatic composition according to the invention, said container being hermetically closed and suitable for preserving the sterility of the enzymatic composition,
a device for introducing the amount of blood plasma medium into the container,
a device for collecting the plasmin-rich ex-vivo plasma medium comprising the enzymatic composition, and
the filter. Advantageously, the filter is a filter for sterilizing the plasmin-rich ex-vivo plasma medium and for forming a sterile plasmin-rich ex-vivo plasma medium free of enzymatic composition.

In a first embodiment of a device according to the invention, each of the constituent components of the kit is separately packaged in a sterile manner in individual packaging.

In a second embodiment of a device according to the invention, some of the constituent components of the kit are packaged together in a sterile manner in sterile form in a common packaging. They may be in assembled or disassembled form or in a partially assembled form and in sterile form.

In a third embodiment of a device according to the invention, some of the constituent components of the kit with the exception of the container containing the enzymatic composition are packaged together—in assembled form or in dissociated form or in a partially assembled form—in non-sterile form in a common packaging and are then sterilized via any known and suitable sterilization process. The container containing the enzymatic composition may be sterilized via any sterilization process that does not harm the enzymatic activity of the immobilized enzyme.

Advantageously and according to the invention, the device for introducing the amount of blood plasma medium into the container comprises:
a syringe comprising a plunger sliding in a cylinder having a dispensing exiting end;
a needle suitable for connecting to the exiting end of the syringe; the syringe and the needle being suitable for cooperating and for allowing:
   collection of an amount of blood plasma medium in a blood plasma medium preparation tube; and
   introduction of said amount of blood plasma medium into the container.

Advantageously, the blood plasma medium preparation tube is also a blood sample collection tube, for example in the form of a Vacutainer® tube (BD Diagnostics, Pont-de-Claix, France), suitable for allowing said sample collection and, where appropriate, for preventing coagulation of said collected blood plasma medium.

Advantageously, the needle of the introduction device has a length suitable for allowing the collection of the blood plasma medium in the blood plasma medium collection and preparation tube.

Advantageously and according to the invention, the sample collection device comprises:
a sterile syringe for collecting an amount of plasmin-rich ex-vivo plasma medium in the container;
the filter suitable for insertion between the sterile syringe and a needle for sample collection from the container of the plasmin-rich ex-vivo plasma medium comprising the enzymatic composition, said filter being capable of receiving the plasmin-rich ex-vivo plasma medium and the enzymatic composition, of retaining the enzymatic composition and of delivering into the syringe plasmin-rich ex-vivo plasma medium free of enzymatic composition.

Advantageously and according to another embodiment of the invention, the device may also comprise an additional sterile needle for dispensing the plasmin-rich blood plasma medium in a patient's body, in the context of the treatment of a cardiovascular pathology or in the context of a surgical intervention, in particular during an intravitreal intervention of a treatment—especially during a surgical intervention—in ophthalmology.

Advantageously and according to the invention, the container is a bottle equipped with a stopper formed from polymer that is able to be pierced with a needle and to allow introduction of the blood plasma medium into the container and collection of the plasmin-rich blood plasma medium from the container.

Advantageously and according to the invention, the filter is a sterilizing filter with a cut-off threshold of less than or equal to 0.22 μm, i.e. a filter suitable for retaining particles whose mean diameter is greater than 0.22 μm, especially bacteria, yeasts and fungi.

Advantageously and according to the invention, the device comprises a sterile outer packaging envelope containing at least the sterile container comprising the sterile enzymatic composition, at least one sterile syringe, at least one sterile needle and the sterile filtration device. Advantageously, the kit formed from the syringe, the needle, the filtration device and the sterile outer packaging envelope may be sterilized via a sterilization treatment (irradiation, ethylene oxide, or the like . . . ) and then combined with the sterile container comprising the enzymatic composition.

The invention also relates to an enzymatic composition, to a preparation process, to the use of such an enzymatic composition and to a device or kit for treating a blood plasma medium, characterized in combination by all or some of the characteristics mentioned hereinabove or hereinbelow.

Other aims, characteristics and advantages of the invention will be apparent upon reading the following description referring to the single figure illustrating a device according to the invention and to the illustrative examples of the invention which are given as a non-limiting guide.

Determination of the Plasminogenase (Urokinase) Activity

The enzymatic activity of a solution comprising a plasminogenase (U/mL) is determined by measuring the initial rate of the hydrolysis reaction at a predetermined temperature of the substrate S-2251 (H-D-Val-Leu-Lys-pNA.2HCl, Chromogenix, Werfen France, Le Pré-Saint-Gervais, France) introduced into the solution.

A measuring medium at 37° C. is formed in a spectrophotometric measuring cuvette by mixing a volume of a solution of plasminogenase in physiological saline and the same volume of an aqueous solution of S-2251 at a concentration of about 2 mM (so that the concentration of S-2251 in the measuring medium is about 1 mM) which is capable of releasing para-nitroaniline (c 10 000 M⁻¹.cm⁻¹) under the action of plasminogenase/urokinase. Starting from the time of mixing, the change in absorbance (optical density, OD₄₀₅ₙₘ) at 405 nm of the measuring medium at 37° C. is measured, for example continuously. The slope at the origin of the curve of change in absorbance at 405 nm, i.e. the initial rate Vi of the reaction expressed in Δ_{abs}/min, is evaluated. The enzymatic activity (expressed in U/mL of measuring medium) is given by formula (I) below:

$$plasminogenase activity = \frac{Vi * Vm * {10}^{3}}{ɛ * L * Vs} , \qquad ( I )$$

in which:
Vi is the initial rate of the reaction expressed in Δ_{abs}/min;
Vm is the volume (in mL) of the measuring medium;
ϵ is the molar extinction coefficient (in M⁻¹.cm⁻¹) of para-nitroaniline at 405 nm;
L is the optical path length (in cm) of the spectrophotometric measuring cuvette; and
Vs is the volume (in mL) of the plasminogenase solution introduced into the spectrophotometric measuring cuvette.

Determination of the Plasmin Activity of a Plasmin-Rich Ex-Vivo Plasma Medium

The enzymatic activity of the plasmin (named plasmin activity) of a plasmin-rich ex-vivo plasma medium free of enzymatic composition and obtained by placing in contact at 37° C. an amount of enzymatic composition according to the invention with an amount of blood plasma medium comprising plasminogen for a time of between 5 minutes and 60 minutes is determined by spectrophotometric measurement of the formation of para-nitroaniline from S-2251.

A measuring medium is prepared in a spectrophotometric measuring cuvette by mixing at 37° C. a volume of plasmin-rich ex-vivo plasma medium and this same volume of an aqueous solution of S-2251 (H-D-Val-Leu-Lys-pNA.2HCl, Chromogenix, Werfen France, Le Pré-Saint-Gervais, France) at a concentration of about 2 mM (so that the concentration of S-2251 in the measuring medium is about 1 mM) which is capable of releasing para-nitroaniline under the action of the plasmin of the plasmin-rich ex-vivo plasma medium. Starting from the time of mixing, the change in absorbance (optical density, OD₄₀₅ₙₘ) at 405 nm of the measuring medium at 37° C. is measured over 5 minutes. The slope at the origin of the curve of change in absorbance at 405 nm, i.e. the initial rate Vi of the reaction expressed in Δ_{abs}/min, is evaluated. The plasmin activity of the plasmin-rich ex-vivo plasma medium (expressed in U/mL of plasmin-rich ex-vivo plasma medium) is given by formula (II) below:

$$plasmin activity = \frac{Vi * Vm * {10}^{3}}{ɛ * L * Vp} , \qquad ( II )$$

in which:
Vi is the initial rate of the reaction expressed in Δ_{abs}/min;
Vm is the volume (in mL) of the measuring medium;
ϵ is the molar extinction coefficient (in M⁻¹.cm⁻¹) of para-nitroaniline at 405 nm;
L is the optical path length (in cm) of the spectrophotometric measuring cuvette; and
Vp is the volume (in mL) of the plasmin-rich ex-vivo plasma medium introduced into the spectrophotometric measuring cuvette.

Determination of the amount of plasminogenase—especially of urokinase—of a plasmin-rich ex-vivo plasma medium

The amount of plasminogenase of a plasmin-rich ex-vivo plasma medium is determined by fluorescence measurement according to any method known per se, for example by assay via the “ELISA” immunoenzymatic technique using a primary antibody specific for plasminogenase (in particular an antibody directed against human urokinase, for example the rabbit antibody ABcam ab24121) and a quantifiable secondary antibody (for example a goat antibody directed against rabbit IgGs and conjugated with HRP (horseradish peroxidase) in the presence of Amplex® UltraRed. A calibration curve is established using solutions containing known amounts of plasminogenase in blood plasma medium.

Preparation of an Enzymatic Composition According to the Invention

In a process for manufacturing an enzymatic composition according to the invention, a solid support in divided form is chosen from the group of supports formed from a porous hydrophilic material, especially from the group of supports formed from particles of a porous hydrophilic material bearing surface grafting groups of epoxide nature. The Epoxy-GE Healthcare support (GE Healthcare Epoxy-Activated Sepharose™) may be chosen as solid support. The support Sepabeads® EC-EP/S (Resindion, Binasca, Italy) may also be chosen as solid support.

The solid support in divided form Sepabeads® EC-HFA/S (Resindion, Binasca, Italy) may be chosen, the mean particle diameter of which is between 100 μm and 300 μm. The particles of the material Sepabeads® EC-HFA/S are formed from polymethacrylate and surface-functionalized with amino-epoxide groups of formula (III) below:

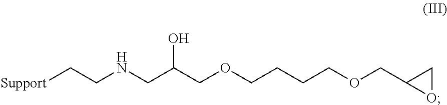

in a proportion of at least 75 μmol of amino-epoxide groups per gram of support in dry form. The mean porosity of the support is between 10 nm and 20 nm.

In a process according to the invention, a plasminogenase—for example a urokinase, a streptokinase or a nattokinase, or a tissue plasminogen activator (t-PA)—is immobilized on a solid material in divided form. To do this, an amount of dry solid material is hydrated in an aqueous hydration composition. The aqueous hydration composition may be, for example, osmosed water, “injection-grade” water for injection (WFI) or sterile physiological saline. For example, 0.2 g of dehydrated solid material in divided form—for example 0.2 g of Sepabeads® EC-HFA/S in dry form—is placed in 40 mL of WFI-grade water for 1 hour at room temperature, and the hydrated solid material is then rinsed three times successively with 0.7 mL of sterile physiological saline at pH 6.8.

The rinsed solid material is then placed in contact with 0.7 mL of an aqueous solution—especially of apyrogenic sterile physiological saline or a BSS (Bioaqua® “Balanced Salt Solution”) intraocular irrigation sterile solution—of human urokinase (U0633, Sigma-Aldrich, Lyon, France) comprising about 2 units (2 U/mL) of plasminogenase activity per mL.

Contact is maintained between the solid support and the enzyme for several hours. The liquid reaction supernatant is collected, and the enzymatic composition thus obtained is then rinsed three times with 0.7 mL of a 1 M solution of NaCl in WFI-grade water or, preferably, sterile physiological saline.

Enzymatic Composition Under GMP Conditions

According to an advantageous embodiment of the invention, synthesis of the enzymatic composition is performed under conditions suitable for forming an enzymatic composition with a content of pyrogenic compounds below the upper acceptable limit value for an injectable composition, especially less than 0.5 endotoxin unit EU/mL. According to this embodiment, the solid support in divided form is a Sepabeads® EC-HFA/S support obtained according to a process complying with good manufacturing practice (GMP) and having a reduced content of pyrogenic endotoxins.

The consumables used, especially the “falcon” tubes, the syringes, the 0.22 μm filters, the micropipette tips and the tubes are consumables certified as being sterile and apyrogenic. The glassware and the laboratory equipment (flask for hydration of the support, lyophilization flasks, stoppers and spatulas) are treated before use with an alkaline detergent solution (E-toxa Clean, 1%) for 16 hours, rinsed with water and sterilized. All the manipulations are performed in a laminar-flow fume cupboard. Starting reagents and solvents that are apyrogenic are chosen and the steps for preparing the enzymatic composition are performed under optimum sterility conditions.

Urokinase U0633 is immobilized on the solid support in divided form Sepabeads® EC-HFA/S under the GMP immobilization conditions described below. 4.46 g of Sepabeads® EC-HFA/S material (Resindion, produced under GMP conditions) are placed for 1 hour at room temperature in 893 mL of WFI-grade water with stirring in order to hydrate the material. The solid support is then rinsed three times with 15.6 mL of physiological saline, and then placed in contact for several hours with 15.6 mL of urokinase (solution of urokinase U0633 in sterile physiological saline, sterilized by filtration on a filter with a cut-off threshold of less than or equal to 0.22 μm ) at a concentration of 2 U/mL of sterile physiological saline so as to form the enzymatic composition. The enzymatic composition is then rinsed three times with 15.6 mL of physiological saline. The rinsing liquid is removed and 0.2 g aliquots of wet enzymatic composition are sampled in sterile lyophilization flasks. The aliquots of wet resin are lyophilized and then stored at 4° C.

Preparation of a Plasmin-Rich Ex-Vivo Plasma Medium

0.7 mL of blood plasma medium is placed in contact with an aliquot of enzymatic composition obtained above, at 37° C. for a time of between 5 minutes and 60 minutes. The plasmin-rich ex-vivo plasma medium is separated from the enzymatic composition by filtration. The plasmin activity (U/mL of plasmin-rich ex-vivo plasma medium) is measured in the presence of the substrate S-2251, the medium being placed at a temperature of 37° C. An enzymatic activity of about 0.2±0.1 U/mL is observed for a contact time of the blood plasma medium and of the enzymatic composition of between 5 minutes and 60 minutes, especially about 15 minutes.

### EXAMPLE 1

### Preparation of an Enzymatic Composition According to the Invention

0.2 g of “GMP” Sepabeads® EC-HFA/S material is hydrated in WFI-grade water, and the hydrated material is then rinsed three times successively with 0.7 mL of sterile physiological saline at pH 6.8. The material thus rinsed is placed in contact with 0.7 mL of a solution of plasminogenase (urokinase U0633) in physiological saline having an enzymatic activity of 2 U/mL. Contact is maintained between the support and the enzyme for several hours. The liquid reaction supernatant is removed and three successive rinses are then performed.

The mass of urokinase present in the ex-vivo plasma medium obtained by placing in contact 0.2 g of the enzymatic composition and 0.7 mL of blood plasma medium for 60 minutes at a temperature of 37° C., measured via the ELISA immunoenzymatic method, is between 2 μg and 20 μg.

### EXAMPLE 2

### Effect of Lyophilization on the Activity of the Enzymatic Composition According to the Invention

Urokinase is immobilized on a “GMP” Sepabeads EC-HFA/S solid support in divided form via the method described in Example 1. A step of lyophilization of the enzymatic composition obtained is or is not performed. The capacity of the lyophilized or non-lyophilized enzymatic composition is studied by placing the same amount (0.2 g) of enzymatic composition in 0.7 mL of blood plasma medium for a time of 15 minutes or 60 minutes. The plasmin-rich ex-vivo plasma medium and the enzymatic composition are separated by filtration. A solution of the substrate S-2251 at a final concentration of 1 mM in the measuring medium is added to the plasmin-rich ex-vivo plasma medium at 37° C. and the plasmin activity of the ex-vivo plasma medium is measured. The results are given in Table 1 below.

TABLE 1

| Lyophilization of the | Plasmin activity at | Plasmin activity at |
|---|---|---|
| enzymatic composition | 15 minutes, U/mL | 60 minutes, U/mL |
| no | 0.178-0.193 | 0.172-0.178 |
| yes | 0.174-0.196 | 0.191-0.194 |

Lyophilization of the enzymatic composition has no effect on its activity for converting the plasminogen of a blood plasma medium into plasmin.

Sterilization of the Enzymatic Composition by Irradiation

A step of irradiating at 25 kGy the enzymatic composition after lyophilization is performed. The activity of the irradiated enzymatic composition is analysed indirectly by measuring the plasmin activity of a plasmin-rich ex-vivo plasma medium obtained by placing said sterilized enzymatic composition in contact with a blood plasma medium for 15 minutes or 60 minutes. The enzymatic composition is removed by filtration and the plasmin activity of the plasmin-rich ex-vivo plasma media thus obtained is measured in the presence of S-2251. The plasmin activity of the plasmin-rich ex-vivo plasma media obtained after 15 minutes of contact is between 0.116 and 0.148 U/mL and the plasmin activity of the plasmin-rich ex-vivo plasma media obtained after 60 minutes of contact is between 0.200 and 0.218 U/mL. Sterilizing irradiation of the enzymatic composition makes it possible to conserve a plasmin activity of the plasmin-rich ex-vivo plasma medium obtained after 15 minutes of contact which is greater than 0.1 U/mL and a plasmin activity of the plasmin-rich ex-vivo plasma medium obtained after 60 minutes of contact which is greater than 0.2 U/mL.

Assay of the Free Urokinase in the Plasmin-Rich Ex-Vivo Plasma Medium

Assay via the ELISA immunoenzymatic technique of the amount of urokinase present in the plasmin-enriched ex-vivo plasma medium obtained by placing a blood plasma medium in contact with the enzymatic composition for 60 minutes at 37° C. is presented in Table 2 below.

TABLE 2

Enzymatic composition

| Irradiation, kGy | Lyophilization | Free urokinase, μg |
|---|---|---|
| 0 | no | 10.0-13.0 |
| 0 | yes | 7.1-9.1 |
| 25 | yes | 4.3-7 |

Lyophilization coupled with terminal sterilization by irradiation makes it possible to reduce the amount of urokinase released by the enzymatic composition into the plasmin-rich ex-vivo plasma medium.

The amount of free urokinase present in the plasmin-rich ex-vivo plasma medium obtained by placing a blood plasma medium in contact with an enzymatic composition (comprising a urokinase immobilized on a solid support in divided form) subjected to irradiation is less than the amount of urokinase present in a plasmin-rich ex-vivo plasma medium obtained by placing a blood plasma medium in contact with an enzymatic composition that has not been subjected to irradiation. The combination of treatments by irradiation and lyophilization of the enzymatic composition makes it possible to obtain amounts of urokinase in the plasmin-rich ex-vivo plasma medium that are less than or equal to about 10 μg, in particular less than 5 μg.

The enzymatic composition according to the invention makes it possible to convert a blood plasma medium into plasmin-enriched ex-vivo plasma medium with a high plasmin activity. It makes it possible to convert the plasminogen of a blood plasma medium into plasmin without introducing into the ex-vivo plasma medium formed a large amount of immunogenic plasminogenase.

Preparation of a Blood Plasma Medium and Activation

An amount of blood is collected in a sterile manner from a patient to be treated, into a sample collection tube (BD Vacutainer®, BD Diagnostics, Pont-de-Claix, France) comprising an anticoagulant such as EDTA or sodium citrate. A step of separating the blood cells and the blood plasma medium is performed by centrifugation at 4000 rpm for 15 minutes. The blood plasma medium is placed in contact, in a sterile manner, with the enzymatic composition at a temperature of 37° C. for a time of at least 15 minutes necessary to allow the conversion of plasminogen into plasmin. The enzymatic composition and the plasmin-rich ex-vivo plasma medium free of free urokinase are separated by filtration on a sterilizing filter (cut-off threshold of 0.22 μm), for example on a Millex PVDF filter (Millipore) or on an Acrodisk Syringe Filter, PN4602 (Pall). Intraocular injection of a suitable volume of the sterile plasmin-rich ex-vivo plasma medium is then performed.

Assay of Free Urokinase in the Plasmin-Rich Ex-Vivo Plasma Medium

Assay via the ELISA immunoenzymatic method of the amount of free urokinase present in the plasmin-rich ex-vivo plasma medium shows a mean amount of free urokinase of less than 20 μg.

A device 20 according to a particular variant of the invention represented in the single figure comprises:
a hermetically closed container—for example a glass container 1—containing an amount of sterile enzymatic composition 2 according to the invention;
means 3 for introducing an amount of blood plasma medium into the container 1 in a sterile manner and for placing said amount of blood plasma medium in contact with the enzymatic composition 2 and comprising:
   a sterile syringe 4 for dispensing the amount of blood plasma medium from a blood sample collection tube and for introducing said amount of collected blood plasma medium into the container 1, said sterile dispensing syringe 4 comprising a plunger 6 sliding in a cylinder 7 having an exiting axial end, named dispensing end 8;
   a dispensing needle 5 suitable for connecting to the dispensing end 8 of the sterile dispensing syringe 4 and having a pointed end 9 suitable for introduction into the container 1 through a pierceable stopper 10 and for introducing the blood plasma medium into the container 1 under the effect of the translational movement of the plunger 6 sliding in the cylinder 7. The dispensing needle 5 is preferably, for example, a needle of about 20 gauge (outside diameter of 0.9081 mm for a wall thickness of 0.1524 mm);
means 11 for collecting and filtering an amount of plasmin-rich ex-vivo plasma medium under the effect of the enzymatic composition 2, comprising:
   a sterile syringe 12 for preparing an amount of plasmin-rich ex-vivo plasma medium from the container 1;
   a filtration device 13 having a plasmin-rich ex-vivo plasma medium inlet 14, and an outlet 15 that can be connected to the sterile preparation syringe 12 and which is configured to be able to dispense sterile plasmin-rich ex-vivo plasma medium in the sterile preparation syringe 12, the filtration device 13 comprising a filter 16 that is capable of retaining the enzymatic composition of the plasmin-rich ex-vivo plasma medium flowing between the inlet 14 and the outlet 15 under the effect of the sterile preparation syringe 12, said inlet 14 being suitable to be assembled with and connected hermetically to a needle 17 for collecting plasmin-rich ex-vivo plasma medium from the container 1;
   said collection needle 17 being suitable to be placed in blood plasma medium communication with the inlet 14 of the filtration device 13 and having a pointed end 18 suitable for introduction into the container 1 and for collecting plasmin-rich ex-vivo plasma medium from the container 1. The sample collection needle 17 is preferably, for example, a needle of about 20 gauge (outside diameter of 0.9081 mm for a wall thickness of 0.1524 mm).

The pierceable stopper 10 formed from an elastic polymer—for example chlorobutyl—which is suitable for piercing with the needle 5 and for introducing blood plasma medium into the container 1 and for collecting plasmin-rich ex-vivo plasma medium from the container 1.

The filtration device 13 is a device for sterilization by filtration on a filter 16 with a cut-off threshold of about 0.22 μm, i.e. on a filter that is suitable for retaining particles with a mean diameter of greater than 0.22 μm.

The device 20 comprises a sterile outer packaging envelope 30 for the container 1 comprising the enzyme composition, the means 3 for introducing an amount of blood plasma medium into the container 1 in a sterile manner and the means 11 for collecting and filtering the plasmin-rich ex-vivo plasma medium. The sterile outer packaging envelope 30 containing the means 3 for introducing an amount of blood plasma medium into the container 1 (comprising the sterile dispensing syringe 4 and the dispensing needle 5) in a sterile manner and the means 11 for collecting and filtering plasmin-rich ex-vivo plasma medium (comprising the sterile preparation syringe 12, the filtration device 13 and the sample collection needle 17) may be sterilized after packaging via any suitable sterilization means, the hermetically closed container 1 containing the sterile enzymatic composition 2 according to the invention being sterilized via sterilization means that do not harm the functionality of the enzymatic composition 2.

In a variant not shown, the device 20 may also comprise an additional sterile needle for injecting into a patient's body a suitable volume of plasmin-rich ex-vivo plasma medium contained in the sterile syringe 12 for preparing the amount of plasmin-rich ex-vivo plasma medium. Such an injection needle is a needle of between 25 and 30 gauge (i.e. the outside diameter of which is between 0.30 mm and 0.50 mm).

The sterile syringe 12 for preparing the amount of plasmin-rich ex-vivo plasma medium may have an exiting end formed from an adapter of “Luer-lock” type and the injection needle may have an end complementary to the “Luer-lock” adapter of the syringe 12.

A device according to another variant of the invention, not shown, may comprise:
a hermetically closed container 1 containing an amount of sterile enzymatic composition 2 according to the invention;
a sterile syringe 12 for preparing an amount of plasmin-rich ex-vivo plasma medium from the container 1, said sterile syringe 12 being a precision syringe that is capable of containing and delivering a volume of sterile plasmin-rich ex-vivo plasma medium free of enzymatic composition of between 100 μL and 250 μL; and
a filtration device 13.

The invention may be the subject of numerous variants without departing from the scope of protection. For example, the constituent components of a device according to the invention may be in disassembled form or in partially assembled form in the outer envelope 30.

1. Enzymatic composition comprising:
at least one enzyme, named plasminogenase, for converting into plasmin at least some of the plasminogen of a blood plasma medium comprising plasminogen;
a solid support that is insoluble in aqueous solution, the solid support having dimensions suitable for allowing it to be retained on a filter with a cut-off threshold of less than or equal to 0.22 μm;
wherein said plasminogenase is linked to the solid support and remains linked to this support on contact with a blood plasma medium, and in that the composition is in dry form.

2. Composition according to claim 1, wherein the solid support is in divided form and formed from particles having three dimensions extending in three mutually orthogonal directions, at least two of the three dimensions being greater than 0.22 μm.

3. Composition according to claim 1, wherein the solid support is formed from a material chosen from the group formed from polymethacrylic polymers.

4. Composition according to claim 1, wherein at least one plasminogenase is a serine endopeptidase of the class EC 3.4.21 of the enzyme classification.

5. Composition according to claim 1, wherein at least one plasminogenase is linked to the solid support via at least one covalent bond.

6. Composition according to claim 1, wherein it is sterile.

7. Composition according to claim 1, wherein it is in dehydrated powder form.

8. Composition according to claim 1, wherein at least one plasminogenase is linked to the solid support via a group of atoms comprising a main chain of atoms linearly bonded to each other via covalent bonds, the main chain having a number of atoms at least equal to 4.

9. Composition according to claim 1, wherein each plasminogenase is linked to the solid support so as only to introduce into a blood plasma medium with which the enzymatic composition is placed in contact a mass of free plasminogenase of less than 400 μg, said contact being performed according to the process below:
a mass of between 0.01 g and 0.5 g of enzymatic composition in dehydrated form is mixed at a temperature of about 37° C. with a volume of between 0.5 mL and 1.0 mL of blood plasma medium; and then
the contact is maintained for a time of more than 5 minutes; and then
the enzymatic composition and the blood plasma medium are separated by filtration on a filter with a cut-off threshold of less than or equal to 0.22 μm; and
the mass of plasminogenase released into the blood plasma medium is measured.

10. Composition according to claim 1, wherein it has an activity, named plasminogenase activity, for converting into plasmin the plasminogen of a blood plasma medium with which it is placed in contact under the following conditions:
a mass of between 0.01 g and 0.5 g of enzymatic composition in dehydrated form is placed in contact at a temperature of about 37° C. for a time of more than 5 minutes with a volume of between 0.5 mL and 1.0 mL of blood plasma medium, and
a plasmin-rich ex-vivo plasma medium is formed, which has, after separation by filtration of the enzymatic composition and of the plasmin-rich ex-vivo plasma medium, an initial enzymatic activity, named plasmin activity, as measured via a para-nitroaniline release test of greater than 0.1 μmol of para-nitroaniline released per minute and per millilitre (mL) of plasmin-rich ex-vivo plasma medium, said release test consisting in:
mixing in the plasmin-rich ex-vivo plasma medium maintained at a temperature of 37° C. a chromogenic substrate S-2251 of formula (I) below: 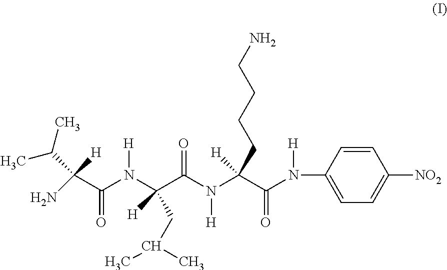 [SMILES: CC(C)C[C@H](NC(=O)[C@H](N)C(C)C)C(=O)N[C@@H](CCCCN)C(=O)Nc1ccc([N+](=O)[O-])cc1]
at an initial concentration of about 1 mM (i.e. 10⁻³ mol/L) in the plasmin-rich ex-vivo plasma medium,
evaluating, following the mixing, the initial rate of para-nitroaniline release (in μmol of para-nitroaniline) per minute and per millilitre (mL) of plasmin-rich ex-vivo plasma medium.

11. Process for preparing an enzymatic composition according to claim 1, in which:
at least one enzyme, named plasminogenase, for converting into plasmin at least some of the plasminogen of a blood plasma medium comprising plasminogen is chosen;
a solid support that is insoluble in aqueous solution is chosen:
which is suitable for forming with each plasminogenase a bond that is stable on contact with a blood plasma medium; and
which has dimensions that are suitable for allowing it to be retained on a filter with a cut-off threshold of less than or equal to 0.22 μm; and
the solid support and each plasminogenase are placed in contact so as to link each plasminogenase to the solid support; and
a lyophilization step is performed so as to form the enzymatic composition.

12. Process according to claim 11, wherein a solid support in divided form is chosen, formed from particles having three dimensions extending in three mutually orthogonal directions, at least two of said three dimensions being greater than 0.22 μm.

13. Process according to claim 11, wherein at least one step of sterilization of the enzymatic composition is performed.

14. Use of an enzymatic composition according to claim 1 for preparing a sterile plasmin-rich ex-vivo plasma medium free of enzymatic composition.

15. Use according to claim 14, wherein an amount of the enzymatic composition is placed and maintained in contact with an amount of the blood plasma medium comprising plasminogen for a time of between 15 minutes and 60 minutes at 37° C., so as to form the plasmin-rich ex-vivo plasma medium comprising a mass of free plasminogenase of less than 25 μg per mL of plasmin-rich ex-vivo plasma medium.

16. Device for preparing a sterile plasmin-rich ex-vivo plasma medium free of enzymatic composition, comprising an amount of enzymatic composition according to claim 1 and a filter with a cut-off threshold of less than or equal to 0.22 μm.

17. Device (20) according to claim 16, further comprising:
a container (1) containing the amount of enzymatic composition;
a device (3) for introducing blood plasma medium into the container (1);
a device (11 ) for collecting from the container (1) a plasma medium formed in the container under the effect of the enzymatic composition;
the sample collection device (11) and the filter being arranged to allow the filtration of the plasma medium and the production of a filtrate constituting a sterile plasmin-rich ex-vivo plasma medium free of enzymatic composition.

18. Composition according to claim 2, wherein the solid support is formed from a material chosen from the group formed from polymethacrylic polymers.

19. Composition according to claim 2, wherein at least one plasminogenase is a serine endopeptidase of the class EC 3.4.21 of the enzyme classification.

20. Composition according to claim 3, wherein at least one plasminogenase is a serine endopeptidase of the class EC 3.4.21 of the enzyme classification.
